(19) United States
(12) Patent Application Publication
(10) Pub. No.: US 2008/0306395 A1
(43) Pub. Date: Dec. 11, 2008

(54) Portable Electrocardiograph

(75) Inventors: Feng Xu, Beijing (CN); Weihu Wang, Beijing (CN)

Correspondence Address: SEYFARTH SHAW LLP, WORLD TRADE CENTER EAST, TWO SEAPORT LANE, SUITE 300, BOSTON, MA, 02210-2028, US

(73) Assignee: Beijing Choice Eletronic Technology Co., Ltd., Beijing (CN)

(21) Appl. No.: 12/159,503

(22) PCT Filed: Feb. 8, 2007
(86) PCT No.: PCT/CN07/00447

§ 371 Date: Jun. 27, 2008

(30) Foreign Application Priority Data
Apr. 25, 2006 (CN) 200610076050.3

(51) Int. Cl. A61B 5/0404

(52) U.S. Cl. 600/509

(57) ABSTRACT

A portable electrocardiograph comprising a housing and a second electrode which is provided on one end of the housing, characterized in that a clamping cover which tends to close automatically is provided on the other end of the housing; an oval finger hole is provided between the clamping cover and the housing; the lower half of the inner wall of the finger hole is defined on the housing; the lower half of the inner wall is provided with a first electrode; and the upper half of the inner wall of the finger hole is provided on the clamping cover, such that the present invention provides the portable electrocardiograph of simple structure, convenient use, and high reliability for heart disease sufferers.

### TECHNICAL FIELD

The present invention relates to a portable electrocardiograph, in particular to a portable electrocardiograph which can simultaneously measure the blood oxygen saturation and the electrocardiogram.

More particularly, the present invention relates to a portable electrocardiograph which can measure the pulse rate by a pair of photoelectric cells when the cardiac rate cannot be correctly measured through electrocardiogram waveform due to excessively large skin impedance or instable contact of another electrode, and at the same time, can independently measure the pulse rate parameter and the blood oxygen saturation parameters, so as to further judge whether the pulse rate and the respiratory function of a patient are normal, thus facilitating the users with heart disease.

### BACKGROUND OF THE INVENTION

In the prior art, a noninvasive blood oxygen saturation is generally measured by a multi-parameter bedside monitor together with a cardiograph and a blood pressure. In additional, the applying electrocardiograph performs continuous measurement. However, abnormal electrocardiogram caused by arrhythmia and the like heart diseases is accident and extremely uneasy to check.

The portable electrocardiograph according to the prior art is convenient for patient to check the heart state at any situation in time, monitor the electrocardiogram waveform in real time, and store the check record. If the skin of user has excessively large impedance or has instable contact with the electrode such that the cardiac rate cannot be correctly measured through electrocardiogram waveform, a pair of photoelectric cells are needed to measure pulse rate, or it is necessary to independently measure the pulse rate parameter and the blood oxygen saturation parameter, so as to further judge whether the pulse rate and the respiratory function of patient are normal.

However, it is hard for a heart disease sufferer to use his/her two hands at the same time to use the portable electrocardiograph and the blood oxygen saturation measurement instrument.

### SUMMARY OF THE INVENTION

The object of the present invention is to provide a portable electrocardiograph which can simultaneously measure the blood oxygen saturation and the electrocardiogram.

Another object of the present invention is to provide a portable electrocardiograph which can measure the pulse rate by a pair of photoelectric cells when the cardiac rate cannot be correctly measured through the electrocardiogram waveform due to excessively large skin impedance or instable contact of another electrode.

The further object of the present invention is to provide a portable electrocardiograph which can independently measure the pulse rate parameter and the blood oxygen saturation parameter and further judge whether the pulse rate and the respiratory function of patient are normal, so as to provide convenience for the heart disease sufferer.

Another object of the present invention is to provide a portable electrocardiograph which is of simple structure, convenient use, and high reliability.

Therefore, according to one aspect of the present invention, there is provided a portable electrocardiograph which comprises a housing and a second electrode which is provided at one end of the housing, characterized in that a clamping cover which can automatically tend to close is provided at the other end of the housing; an oval finger hole is defined between the clamping cover and the housing; the lower half of an inner wall of the finger hole is defined on the housing, and the lower half of the inner wall is provided with a first electrode; and the upper half of the inner wall of the finger hole is defined on the clamping cover.

Preferably, a photoelectric receiving cell is provided in the inner wall of the lower half of the finger hole, and a light emitting diode is provided at the corresponding position of the inner wall of the upper half of the finger hole.

Preferably, the clamping cover is closed with the housing by means of a torsion spring.

Preferably, the torsion spring is sleeved on a shaft which is connected with the housing.

Preferably, the clamping cover is closed with the housing through a U-shaped spring sheet.

Preferably, the first electrode provided on the lower half of the inner wall is of a half-ring shape.

Preferably, the inner wall of the upper half is covered with a half-ring shaped soft material layer.

Preferably, the top face of the photoelectric receiving cell is provided to be at the same surface as the top face of the first electrode, and the top face of the light emitting diode is slightly indented into the surface of the soft material layer.

Preferably, the side face, with a display screen, of the housing is of a rectangle, trapezoid, or drum shape.

Preferably, the second electrode is of a straight line or curvilinear shape closely fit with human body.

According to the other aspect of the present invention, there is provided a method for simultaneously measuring the blood oxygen saturation and the electrocardiogram, characterized in that a clamping cover which can automatically tend to close is provided at one end of a housing of the portable electrocardiograph; an oval finger hole is defined between the clamping cover and the corresponding housing; a first electrode and a pair of photoelectric cells are provided in the inner wall of the finger hole, it is to perform an electrocardiogram measurement by means of the first electrode and a second electrode which is provided on the other end of the housing; and in the case that the clamping cover is pressurized, the first electrode in the finger hole closely contacts the finger, so that the pulse rate measurement is performed by a pair of photoelectric cells.

According to the present invention, the portable electrocardiograph employs the half oval or semi-circular electrode to contact the finger, thereby increasing contacting area, reducing skin impedance, and improving precision of detection.

The portable electrocardiograph according to the present invention simultaneously employs a pair of photoelectric cells, can measure the pulse rate through a pair of photoelectric cells when the pulse rate cannot be correctly measured through the electrocardiogram waveform due to excessively large skin impedance or instable contact of another electrode, and can independently measure the pulse rate parameter and the blood oxygen saturation parameter, so as to further judge whether the pulse rate and the respiratory function of patient are normal.

The portable electrocardiograph according to the present invention employs the first electrode and the second electrode which is provided on the other end of the housing, in order to realize electrocardiogram measurement. In the case that the clamping cover is pressurized, the first electrode in the finger hole closely contacts the finger, thereby increasing contacting area, reducing skin impedance, and improving precision of detection. Simultaneously, a pair of photoelectric cells is used to measure the pulse rate and the blood oxygen saturation. The pair of photoelectric cells can be used to measure the pulse rate when the cardiac rate cannot be correctly measured through the electrocardiogram waveform due to excessively large skin impedance or instable contact of another electrode, and it can independently measure the pulse rate parameter and the blood oxygen saturation parameter, so as to further judge whether the pulse rate and the respiratory function of patient are normal, thus providing convenience for the heart disease sufferer.

### BRIEF DESCRIPTION OF THE ACCOMPANYING DRAWINGS

FIG. 1 is a perspective view of the portable electrocardiograph according to the present invention;

FIG. 2 is a section view of internal structure of the portable electrocardiograph according to the present invention;

FIG. 3 is a section view of internal structure of the portable electrocardiograph along A-A line shown in FIG. 2 according to the present invention.

FIG. 4 is a schematic view of the end face of the finger hole of the portable electrocardiograph according to the present invention; and

FIG. 5 is a schematic view of the end face of the second electrode of the portable electrocardiograph according to the present invention.

### DETAILED DESCRIPTION OF THE PREFERRED EMBODIMENTS

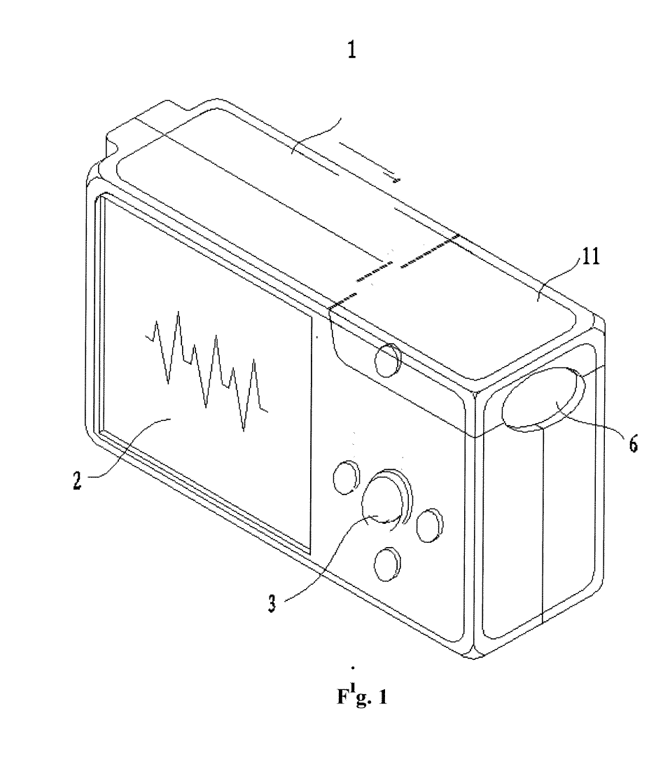

Next, the embodiments of the present invention are described in more detail in combination with FIG. 1 to FIG. 5.

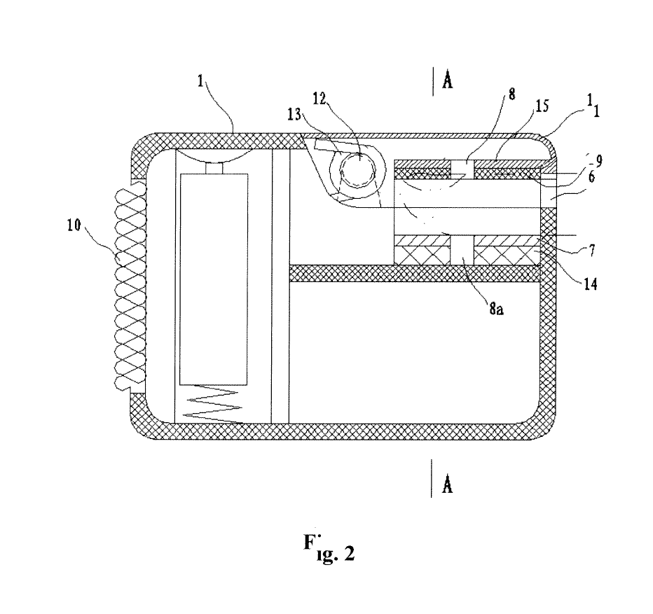

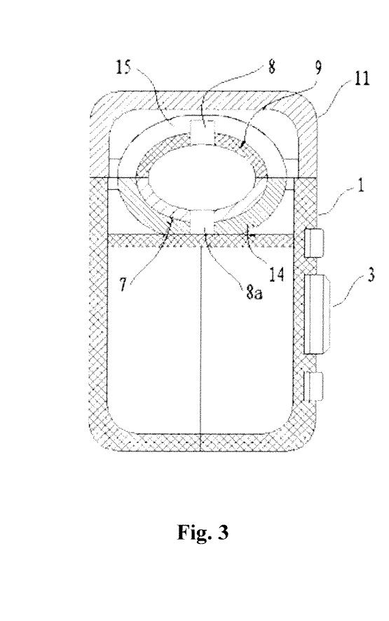

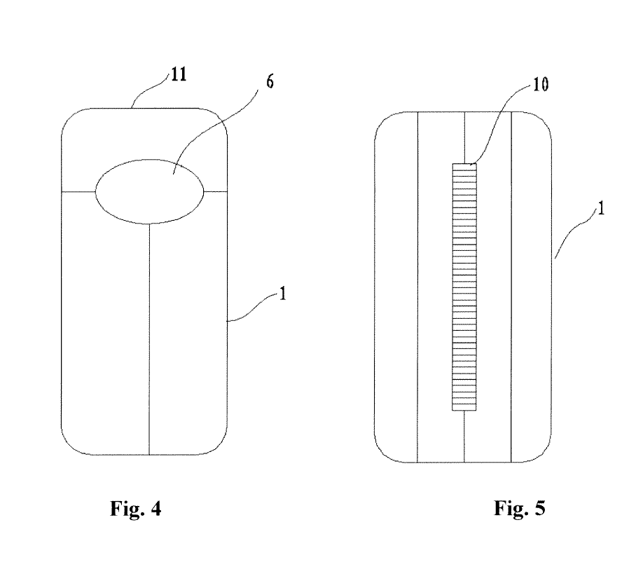

FIG. 1 is a perspective view of one embodiment according to the present invention; FIG. 2 is a section view of internal structure of the embodiment; FIG. 3 is a section view of internal structure of the embodiment along A-A line shown in FIG. 2; FIG. 4 is a schematic view of the end face of the finger hole of the embodiment; and FIG. 5 is a schematic view of the end face of the second electrode of the embodiment.

In the embodiment, a housing 1 is of a cuboid shape. A second electrode 10 (shown in FIG. 2 and FIG. 5) is provided on the left end face (shown in FIG. 2) of the housing 1. For achieving operation of instrument and the display of electrocardiogram waveform, a keyboard 3 (shown in FIG. 1) and a display 2 are provided on a larger side face of the housing 1.

A clamping cover 11 is provided on one corner of the right end face of the housing 1 along the longitudinal direction of the housing 1. A shaft 12 is fixed on the housing 1. The clamping cover 11 is fit on the housing 1 by means of a torsion spring 13 mounted on the shaft 12, such that the clamping cover 11 can automatically close when no external force is exerted to lift the clamping cover (shown in FIG. 2). The torsion spring 12 enables the clamping cover 11 tend to rotate around the axis of the shaft 12, such that the clamping cover 11 is in the best finger-clipping state.

The fitting between the clamping cover 11 and the housing 1 is not in this only way, and a U-shaped spring sheet can also be used to realize automatic close of the clamping cover 11.

An oval finger hole 6 (shown in FIG. 4) is provided to open between the clamping cover 11 and the corresponding portion of the housing 1. A lower half 14 of the inner wall of the finger hole is defined on the housing 1, and an upper half 15 thereof is defined on the clamping cover 11. The depth of the finger hole 6 is about a size as long as for inserting the front one to two knuckles of a finger into the hole. The inner wall of the hole 6 should be made of insulation material. A layer of half-ring shaped good conductive material is provided to cover the surface of the inner wall of the lower half 14 of the finger hole 6 as a first electrode 7, and a layer of half-ring shaped soft material layer 9 (shown in FIG. 2 and FIG. 3) is provided to cover the inner wall of the upper half 15 of the finger hole 6.

To measure the electrocardiogram, the user can first lift the clamping cover 11, then inserting the forefinger or middle finger into the finger hole 6 such that the finger can closely contact the first electrode 7 as the clamping cover 11 is automatically closed by pressure, and in this situation, making a second electrode 10 on the left end face closely contact the user's skin of his/her chest.

In the present invention, a pair of photoelectric cells 8 and 8a are fit in the inner wall of the finger hole 6. This pair of photoelectric cells is oppositely fit in the inner wall of the finger hole 6. A light emitting diode 8 is fit on the upper half 15 of the finger hole 6, and a photoelectric receiving cell 8a is fit on the lower half 14 of the finger hole 6. The ray emitted from the light emitting diode 8 is generated to pass the finger inserted into the finger hole 6 and is received by the photoelectric receiving cell 8a on the opposite wall. To prevent insertion of finger from damaging this pair of photoelectric cells, the top face of the photoelectric receiving cell 8a is provided to be in the same surface as the inner surface of the first electrode 7, and the top face of the light emitting diode 8 should be slightly indented into the surface of the soft material layer 9. In the embodiment, the light emitting diode 8 is fit on the upper half 15 of the finger hole 6; and the photoelectric receiving cell 8a is fit on the lower half 14 of the finger hole 6 (shown in FIG. 2 and FIG. 3). Actually, the portable electrocardiograph also works if the pair of photoelectric cells 8 and 8a exchange their locations.

This pair of photoelectric cells can measure the pulse rate and the blood oxygen saturation, making the portable electrocardiograph is more versatile. As this pair of photoelectric cells is required to keep as short distance as possible with finger when the blood oxygen saturation is measured. Therefore, the clamping cover is specially designed to guarantee keeping an appropriate pressure for finger during measurement.

In the embodiment, the housing is of a cuboid shape, however, it is not the exclusive shape, and other shapes may also be used likewise. The side face having the display of the housing can be of a rectangle, trapezoid, or drum shape.

A pair of photoelectric cells is used in the embodiment. Likewise, it does not means that only one pair of photoelectric cells can be used.

In another embodiment, the second electrode may not be a straight line, but a curvilinear shape closely fit with human body, so as to further improve the effect of measurement.

In another embodiment, the side surface of the clamping cover is provided with an upper marking line which is transversely aligned with the light emitting diode; the housing is provided with a lower marking line which is transversely aligned with the photoelectric receiving cell; and the upper marking line and the lower marking line are vertically aligned. Thus, no matter the finger of user is long or short, and thick or thin, the finger can be put into a suitable position through intuitionistic marks, so as to further improve accuracy of measurement.

The portable electrocardiograph according to the present invention has the advantages of multiple functions, convenient use, and accurate measurement, in which measurement of both the pulse rate and the blood oxygen saturation further make the portable electrocardiograph to be a low cost portable checking instrument for patients.

The present invention is described as above in detail according to a plurality of embodiments. However, those skilled in the art should understand that various modifications and improvements can be made on the present invention, not departing from the spirit of the present invention, and the scope of protection is defined by the attached claims of the present invention.

1. A portable electrocardiograph comprising a housing (1) and a second electrode (10) which is provided on one end of the housing (1), characterized in that
a clamping cover (11) which automatically tends to close is provided on the other end of the housing (1); an oval finger hole (6) is provided between the clamping cover (11) and the housing (1); a lower half (14) of the inner wall of the finger hole (6) is defined on the housing (1); the lower half (14) of the inner wall is provided with a first electrode (7); and an upper half (15) of the inner wall of the finger hole (6) is defined on the clamping cover (11).

2. The portable electrocardiograph according to claim 1, characterized in that a photoelectric receiving cell (8a) is provided in the inner wall of the lower half (14) of the finger hole (6); and a light emitting diode (8) is provided at the corresponding position of the inner wall of the upper half (15) of the finger hole (6).

3. The portable electrocardiograph according to claim 2, characterized in that the clamping cover (11) is closed with the housing (1) by means of a torsion spring (13).

4. The portable electrocardiograph according to claim 1, characterized in that the torsion spring (13) is sleeved on a shaft (12) which is connected with the housing (1).

5. The portable electrocardiograph according to claim 1, characterized in that the clamping cover (11) is closed with the housing (1) by means of a U-shaped spring sheet.

6. The portable electrocardiograph according to claim 1, characterized in that the first electrode (7) on the lower half (14) of the inner wall is of a half-ring shape.

7. The portable electrocardiograph according to claim 1, characterized in that a half-ring shaped soft material layer (9) is provided to cover the inner wall of the upper half (15).

8. The portable electrocardiograph according to claim 1, characterized in that the top face of the photoelectric receiving cell (8a) is provided to be at the same surface as the top face of the first electrode (7); and the top face of the light emitting diode (8) is slightly indented into the surface of the soft material layer (9).

9. The portable electrocardiograph according to claim 1, characterized in that the side face having the display of the housing is of a rectangle, trapezoid, or drum shape.

10. The portable electrocardiograph according to claim 1, characterized in that the second electrode is of straight line or curvilinear shape closely fit with human body.
